(19) United States
(12) Patent Application Publication
(10) Pub. No.: US 2008/0227197 A1
(43) Pub. Date: Sep. 18, 2008

(54) ALLOGENEIC STEM CELL BANK

(75) Inventors: Robert Chow, Arcadia, CA (US); Lawrence D. Petz, Tarzana, CA (US); Rubio R. Punzalan, Torrance, CA (US); Denis O. Rodgerson, Malibu, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: STEMCYTE, INC., COVINA, CA (US)

(21) Appl. No.: 12/110,850

(22) Filed: Apr. 28, 2008

Related U.S. Application Data
(63) Continuation of application No. 10/367,339, filed on Feb. 13, 2003.
(60) Provisional application No. 60/357,308, filed on Feb. 14, 2002.

(51) Int. Cl. C12N 5/08

(52) U.S. Cl. 435/372

(57) ABSTRACT

This invention provides methods for supplying a therapy for individuals exposed to radiation following a nuclear event, through the prospective establishment of an undesignated allogeneic stem cell bank with prospective HLA typing of healthy potential recipients.

### CROSS-REFERENCES TO RELATED APPLICATIONS

This application is a continuation of application Ser. No. 10/367,339, filed Feb. 13, 2003, which related to U.S. Provisional Application No. 60/357,308, filed Feb. 14, 2002, the contents of which are incorporated herein by reference in their entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The threat of nuclear attack is no longer remote. Preparation for a nuclear attack in this country is in progress. Increased security measures around nuclear power plants, improved intelligence capabilities of the government, and training for military personnel in case of attack are underway. Threat scenarios for an attack are being explored. Possibilities include dirty bombs, jets or heavy munitions crashing into nuclear reactors, or nuclear devices used on American military in case of war against a country with nuclear capabilities. Despite the awareness that a nuclear attack is a real possibility, there exists no method for treating potential radiation exposure victims of such an attack.

Current stem cell banks are ill prepared for the ramifications of potential exposure of large numbers of civilian or military personnel to lethal irradiation in the event of a nuclear attack. Radiation exposure caused by a nuclear event causes death within weeks by permanent destruction of the bone marrow. The only method by which bone marrow of lethally irradiated people can be restored is by a stem cell transplantation. It is currently impossible to satisfy the need for stem cells in a large scale emergency from pre-existing bone marrow or peripheral blood donor banks. Adequate numbers of stem cells for transplantation of a large numbers of individuals simply do not exist. This invention addresses this and other needs by providing a large racially diverse umbilical cord blood bank and methods of providing stem cells for individuals for immediate transplantation after an attack.

### BRIEF SUMMARY OF THE INVENTION

The current invention provides a cell bank and insurance model to effectively protect members of the population from the devastating effects of exposure to lethal irradiation.

As such, in one embodiment, this invention provides a method for providing a stem cell unit in an allogeneic stem cell bank for a potential recipient. This method includes the following. First, this method provides a plurality of stem cell units which have been typed. The typed stem cell units of this invention form an allogeneic stem cell bank. Second, this method provides a record for each typed stem cell unit in the stem cell bank. Third, this method provides typing for a potential recipient of a stem cell unit and provides each potential recipient with a type identifier. Fourth, this method comprises storing the record for each typed stem cell unit and each type identifier in a database. Fifth, this method further comprises a comparison step whereby the type identifier is compared with each record for each typed stem cell unit to find a matched stem cell unit. And sixth, this invention provides a method for storing a matched stem cell unit in a database for a potential recipient's use, thereby providing a stem cell unit for a potential recipient. Preferably, the stem cell bank or depository or storage facility is a place where stem cells are kept for safe keeping. In some embodiments, the storage facility might be underground, underwater, in caves or in silos. In other embodiments, it may be on the side of a mountain, in submarines or in outer space. The storage facility may be encased in a shielding material such as lead.

In another embodiment, the present invention relates to a method for supplying a stem cell unit to an individual exposed to radiation following a nuclear event, the method comprising: establishing an undesignated radiation protected allogeneic stem cell bank with prospective HLA typing of healthy potential recipients, thereby supplying a stem cell unit to an individual exposed to radiation following a nuclear event.

In one aspect, the invention provides a method for providing a stem cell unit in an allogeneic stem cell bank for many potential recipients.

In one specific embodiment of this invention, a recipient is treated with a matching stem cell unit, when for example, a potential recipient has been exposed to a lethal dose of radiation. In another aspect, many potential recipients have been exposed to lethal doses of radiation. In one embodiment, treatment with the matching stem cell unit occurs immediately after exposure to the lethal dose of radiation.

In one aspect of the invention, the stem cell units comprise hematopoietic and/or non-hematopoietic stem cells or combinations thereof. In another aspect, the stem cell units comprise hematopoietic stem cells.

In one embodiment of the invention, the hematopoietic stem cells comprise long-term hematopoietic stem cells. In one aspect, the hematopoietic stem cells are obtained from bone marrow. In another aspect, the hematopoietic stem cells are obtained from peripheral blood. In yet another aspect, the hematopoietic stem cells are obtained from umbilical cord blood. In still yet another aspect, the hematopoietic stem cells are obtained from placental blood.

In a particular embodiment of the invention, the stem cell units are cryogenically preserved, such as with DMSO.

In one embodiment of the invention, the stem cell units comprise non hematopoietic stem cells. In one aspect, the non-hematopoietic stem cells are capable of converting to hematopoietic stem cells.

In one particular embodiment of the invention, each of the non-hematopoietic stem cells comprise adult stem cells. In another embodiment, each of said non-hematopoietic stem cells comprise embryonic stem cells.

In one aspect of the invention, the invention provides a computer-readable medium or combination of computer-readable media, containing a program for maintaining type information and providing a matched stem cell units for a potential recipient. This program contains code to effect the following. First, it provides a record of typed stem cell units in an allogeneic stem cell bank. Second, it provides a type identifier for a potential recipient. Third, it stores the type identifier and a record of typed stem cell units. Fourth, it compares the type identifier with the record of typed stem cell units to find a matched stem cell unit. Fifth, it stores the matched stem cell unit for the potential recipient's use.

In one aspect of the invention, the medium or media of claim are selected from the group consisting of a RAM, a ROM, a disk, an ASIC, and a PROM.

These and other aspects of the invention will become more apparent when read with the detailed description and figures which follow.

### BRIEF DESCRIPTION OF THE DRAWINGS

FIG. 1 is a flowchart embodying a process of populating a stem cell bank. It includes obtaining stem cell products from individuals, processing the stem cell products, typing the stem cell products, recording identifying information about the stem cell products, and storing the stem cell products in a facility.

FIG. 2 is a flowchart embodying a method of obtaining umbilical cord blood and placental blood from a donor.

FIG. 3 is an expansion of FIG. 2. FIG. 3 explains steps that may be taken when obtaining umbilical cord blood and placental blood from a donor.

FIG. 4 is a flowchart embodying a process of providing particular stem cell products or units for an individual in case of a catastrophic event. It includes obtaining a biological sample from an individual, typing the sample, recording the sample information, matching identifying information associated with the sample with identifying information associated with stored stem cell products and providing a particular matching stem cell unit for potential future use. It may also include transporting and using a stem cell unit for transplantation.

### DETAILED DESCRIPTION OF THE INVENTION

### I. Introduction

This invention relates to methods for providing stem cells for immediate transplantation to a group of people (e.g., the military) in need of immediate treatment. This invention also includes methods of providing stem cells for transplantation to the general population at large.

In this invention, stem cells products are collected from a genetically, racially and ethnically diverse population. After collection, the stem cells products are processed, typed and stored in units in a quick and cost effective manner. A record is provided for each unit, thereby creating an allogeneic stem cell bank. The units are then matched to unrelated individuals, not yet in need of transplantation, who have provided a biological sample. The matching units are available for the individual's use in case of lethal irradiation from a nuclear attack.

The present invention provides a stem cell model for unrelated healthy individuals which can satisfy the need for stem cells in the event of a large scale emergency, such as a nuclear attack. The existing stem cell banks are ill prepared to meet the needs of the population in case of such an emergency. With the advent of this invention, the population can be protected from the devastating effects of a nuclear attack. In addition, this invention provides a method of collecting and storing stem cells from a diverse population that can be made available within days to supply the nation's needs in case of nuclear attack.

### II. Definitions

The term “stem cell” refers to any cells that have the ability to divide for indefinite periods of time and to give rise to specialized cells. Stem cells emanate from all germinal layers (ectoderm, mesoderm and endoderm). Typical sources of stem cells include embryos, bone marrow, peripheral blood, umbilical cord blood, and placental blood. Stem cells can be pluripotent, meaning that they are capable of generating most tissue on an organism. For example, pluripotent stem cells can give rise to cells of the skin, liver, blood, muscle, bone, and the like.

The term “allogeneic” refers to cells, tissue, or organisms that are of different genetic constitution.

The term “long term stem cells” refers more specifically to those stem cells that are capable of self-renewal over indefinite periods of time.

The term “type or “typing” as used herein refers to any and all characteristics of a sample, e.g., stem cell product sample, which might be of relevance or importance for any potential use of the sample. The term and the corresponding testing conducted to determine the “type” of the sample is thus not limited to any particular tests mentioned herein, e.g., HLA typing. Determination of which tests are relevant and how to perform them is entirely conventional and will change with technological developments. Thus the term “type identifier” refers to any characteristic that can be used for identification purposes.

The term “matching” refers to the degree of similarity between the genetic makeup of the stem cell product or unit to be transplanted into an individual and the individual's genetic makeup. For the purposes of this invention, when two people share a type, they are said to be a match meaning that their tissues are immunologically compatible with each other. The degree to which blood parameters need be identical will vary from patient to patient, and from year to year depending on the current state of technology. Matching then refers to providing the desired degree of match. For example, bone marrow and peripheral blood stem cell transplantation requires a greater degree of matching than blood cord stem cell transplantation. Matching can refer to a match with about 90%, 80%, 70%, 60%, 60%, or 40% similarity. A matching stem cell unit is one that is from a donor not related to the potential recipient.

A stem cell bank or depository or storage facility is a place where stem cells are kept for safe keeping. The storage facility may be designed in such a way that the stem cells are kept safe in the event of a catastrophic event such as a nuclear attack. In some embodiments, the storage facility might be underground, in caves or in silos. In other embodiments, it may be on the side of a mountain or in outer space. The storage facility may be encased in a shielding material such as lead.

The term “stem cell product” refers to any stem cell containing product that, after processing, can be used for transplantation purposes. Transplantation purposes include transplantation of stem cells into an individual after exposure to lethal doses of radiation. Processing refers to any step taken to make the stem cell product suitable for transplantation, e.g., purification.

### III. Providing and Maintaining a Stem Cell Bank

The invention provides methods for acquiring, typing and providing stem cells for the use of specific individuals with matching blood and HLA type in case of a nuclear attack.

In one embodiment of this invention, a depository, e.g., a stem cell bank, is provided for storing stem cell units in anticipation of a possible nuclear attack. The present invention, in some embodiments, relates not only to the provision of such a depository, but also to methods for administering such a depository. The present invention also provides methods of making a stem cell insurance model socially and financially viable.

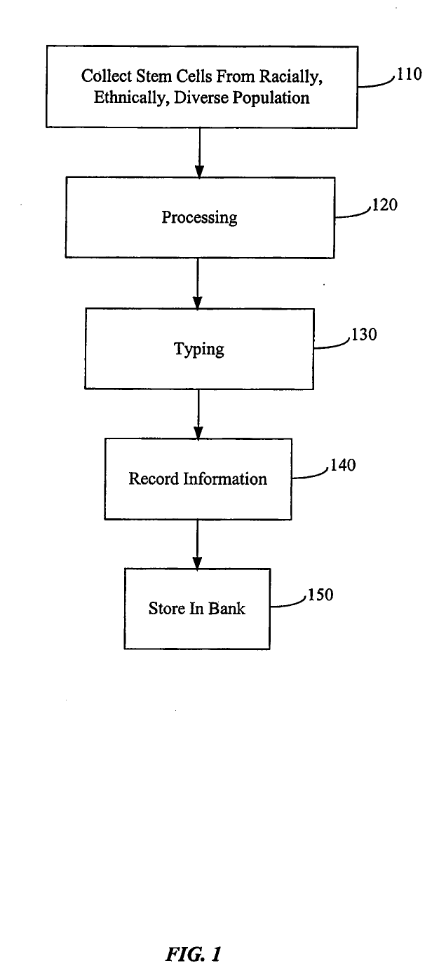

FIG. 1 is one embodiment of a process of acquiring (110), processing (120), typing (130), recording (140) and storing (150) stem cell products in case of lethal radiation exposure of large numbers of persons.

The stem cell facility, e.g., bank, of this invention will comprise stem cell products, e.g., stem cell units, that can be used to treat individuals in case of a catastrophic event such as a nuclear attack. In one particular embodiment of this invention, the stem cell products that comprise the bank will be available to members of the population. With this invention, individuals have available stem cell units suitable for self-transplantation in case of exposure to lethal amounts of radiation.

In order to provide a stem cell bank that can meet the needs of a racially and ethnically diverse population, this invention provides methods of acquiring stem cells from a diverse group of people (110). Once the stem cell products are obtained, this invention provides methods of processing the stem cell products (120). Processing includes any processing method that makes the stem cell products in a form suitable for storage.

This invention also provides methods of typing the stem cell products. Typing includes any method that identifies a stem cell product in such a way that the stem cell unit may be matched to a certain individual. For the purposes of this invention, matching indicates that the stem cell unit is suitable for transplantation into a specific individual.

In a specific embodiment of the invention, the stem cells may be typed using new and improved HLA typing methods. For example, stem cells can be typed using the high-throughput HLA typing-methods described in U.S. Pat. No. 6,670,124, issued Dec. 30, 2003, herein incorporated by reference. A high throughput HLA typing method may include obtaining a biological sample containing template nucleic acid from a subject, amplifying the template nucleic acid with labeled HLA allele-specific primers, hybridizing the amplification products with immobilized HLA locus-specific capture oligonucleotides and using detection methods to determine the HLA genotype of the subject.

This invention also provides methods of recording the stem cell units so that when a stem cell unit needs to be located, it can be easily retrieved (140). Any indexing and retrieval system can be used to fulfill this purpose. This invention also provides a storage system so that the stem cells can be stored (150). In one embodiment, the stem cells will be stored in such way that a storage facility will be able to store hundreds, thousands and even millions of different stem cell units.

A. The Collection and Processing of Stem Cell Products

This present invention includes any known methods of acquiring stem cell products from donors (110). Stem cells products are products that contain stem cells, e.g., umbilical cord blood. Stem cells useful in this invention can be obtained from any stem cell source, e.g., bone marrow, peripheral blood, umbilical cord blood or placental blood. Stem cells of this invention are those that can be used for the treatment of individuals after catastrophic events such as exposure to lethal doses of radiation.

A stem cell donor for the purposes of this invention may include any suitable person willing to donate stem cells for the use of others. In one embodiment, donors are individuals who are in good general health and between the ages of 16 and 60. Certain information may be collected from the donor before or after stem cell donation in order to determine donor suitability. For example, the donor may be given a medical questionnaire to fill out. In one embodiment of this invention, a donor will undergo a medical examination before donation.

Once a suitable donor is found, stem cell products can be collected from the donor. Any known stem cell product collection method can be used in this invention. For example, standard blood bag systems can be used for the collection of stem cell products.

In one embodiment of the present invention, peripheral blood may be collected from a donor. Blood bag systems for the collection and processing of peripheral blood are well known in the art. Standard blood bag systems may consist of a collection bag and a collection needle, connected via a tube. The collection may contain an anticoagulant such as a citrate-phosphate-dextrose solution, citrate-phosphate-dextrose with adenine, sodium citrate solution or an anticoagulant citrate-dextrose solution (ACD) or heparin.

To aid blood processing and improve safety, processing bags for various blood components may be part of a sterile blood bag system. In one embodiment, a red cell storage solution may be incorporated into one of the processing bags. Additionally, both the collection bag and the processing bags may be equipped with ports and break connectors. The ports may be used for the addition or extraction of materials to or from the inside of the bag. A break connector may be used to temporarily close a tube or the entrance of a bag.

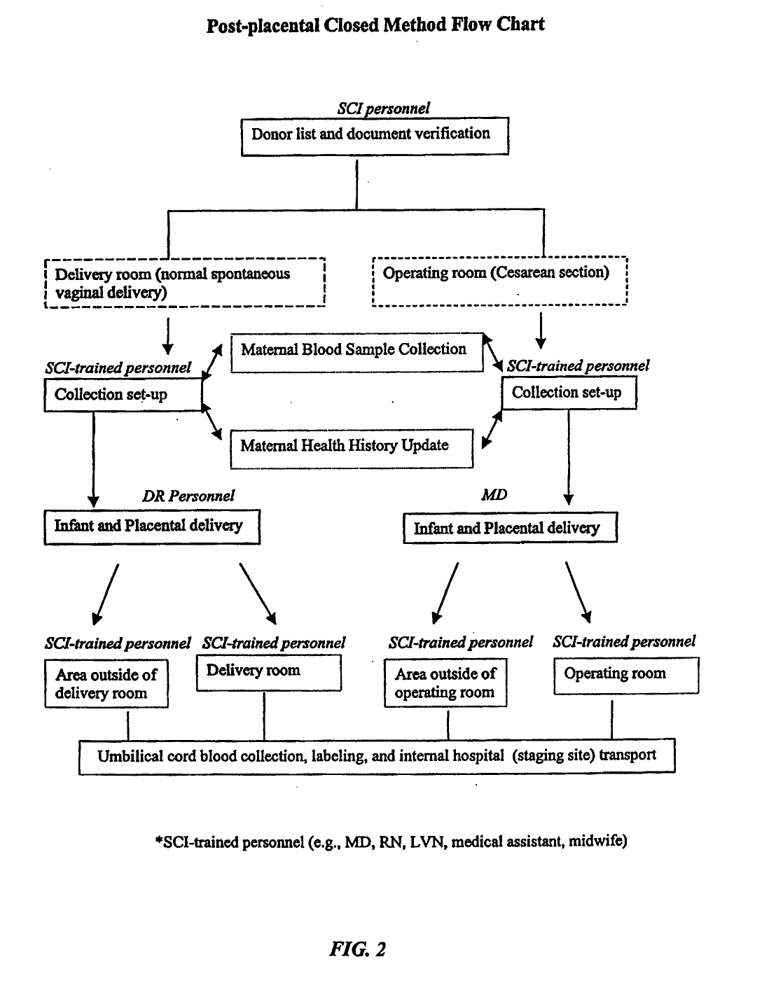

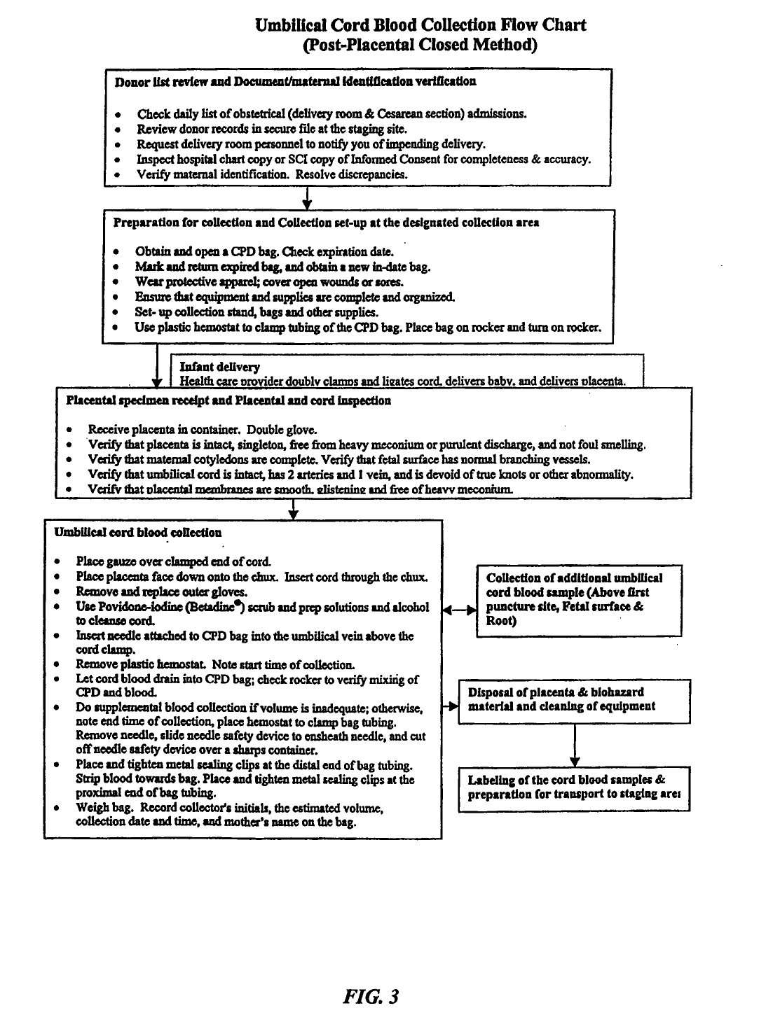

FIG. 2 is a flowchart embodying one method of identifying suitable stem cell donors and collecting stem cell products from them. (FIG. 3 is an expansion of FIG. 2). In this embodiment, postnatal women are asked to donate cord blood and placental blood. Hospitals are contacted and asked to participate in a umbilical cord/placental blood collection project. Potential donors are women who are in labor and about to deliver a baby either by natural delivery or Cesarean section. In U.S. Pat. No. 5,993,387, herein incorporated by reference, one method of obtaining umbilical cord blood and placental blood from postnatal women is described, e.g., enrolling a family with a bank before a child is born and collecting a fee for the collection and storage of the cord stem cells to be collected after birth.

In some embodiments, hospitals throughout the country are asked to participate in a stem cell collection project. Ideally, stem cell products are collected from individuals from different racial and ethnic groups in order to provide potential matches for a diverse group of individuals. Hospitals asked to participate in the stem cell collection project may service all types of communities, e.g., low income, high income and mixed communities.

In one embodiment of this invention, daily lists of obstetrical admissions from participating hospitals are reviewed for potential donors. At the time of impending delivery, preparation is made for the collection of cord and placenta blood from the donor. Preparation and collection may be performed by any hospital worker or any outside worker trained in collection procedures. Collection procedures may include the use of a collection bag to collect the cord blood. In some embodiments, the collection bag may be specially designed for the collection of cord blood.

In one embodiment, after delivery, the placenta and cord blood are collected and examined. In some embodiments, examination ensures that the stem cell products are suitable for further processing. An examination may include examining the placenta to make sure it is intact, singelton, free from heavy meconium or purulent discharge. The umbilical cord may also be examined to determine that it is intact with 2 arteries, 1 vein and devoid of true knots or other abnormalities.

After collection of the cord blood, e.g., into the collection bag, the samples may be labeled and prepared for further transport.

The stem cell products of this invention may be transported to and from a stem cell storage facility, interim facility or stem cell product processing area by methods known in the art. For example, conventional containers for blood can be used for transport, e.g., electronically controlled containers can be transported by express methods or messengers. In some embodiments, the containers will be controlled by a thermoelectric pump. In these embodiments, the temperature of the container remains essentially constant over long periods of times.

In some embodiments of the invention, after collection, the stem cell products are processed (120) according to methods known in the art (See U.S. Pat. Nos. 6,059,968, 5,879,318 incorporated herein by reference). Processing prepares the stem cell products for storage or for further use.

The stem cell containing blood stored in the stem cell bank can be whole blood or any portion or portions thereof. Like normal blood, stem cell containing blood, e.g., cord or placental blood, has a variety of component entities. These entities can be fractionated or otherwise separated from one another using conventional separation techniques, common in conjunction with normal peripheral blood. Similarly, there also exists conventionally utilized various fractionating procedures in conjunction with bone marrow that can be used in the present invention.

In some embodiments, processing concentrates or isolates the stem cells in the stem cell containing product. One method for the purification of stem cell products, e.g., blood, after collection is by sedimentation of red blood cells. In preferred embodiments, after processing, the processed stem cell product contains a sufficient amount of stem cells for the successful transplantation of an adult patient.

In some embodiments, after processing, a sample of the processed stem cell product can be made available for testing. In one particular embodiment of this invention, cord blood is processed. Ideally, the volume of the cord blood after processing will be sufficient for transplantation purposes, e.g., volume will be equal to or greater than 50 ml. Sufficient for transplantation purposes means that enough stem cells are present in the product to successfully treat a person in need of stem cell transplantation. In some embodiments of this invention, the total nucleated cell count of the processed stem cell product will be measured. In preferred embodiments, it may be equal to or greater than 8×10⁷ cells.

In a specific embodiment of the invention, whole cord blood processing may include the following steps: preparation of tubes and labels, sampling of the cord blood component, centrifugation, plasma depletion, cryocyte bag attachment, cryoprotectant preparation, cryoprotectant addition, product splitting for freezing and sterility/infectious disease testing. In one embodiment of the invention, each stem cell product collected from an individual is associated with a unique identification number.

B. The Typing of Stem Cell Products

In some embodiment of this invention, the collected stem cell products will be further classified or typed according to certain identifying features, e.g., HLA typing (130).

The type information may include genotype or phenotype information. Phenotype information may include any observable or measurable parameter, either at a macroscopic or system level or microscopic or even cellular or molecular level. Genotype information may refer to a specific genetic composition of a specific individual organism, for example, whether an individual organism has one or more specific genetic variants up to all the variations in that individual's genome, for example, whether the individual is a carrier of genetic variations that influence disease or the HLA type of that individual.

In one embodiment of this invention, the stem cell products will be HLA typed. Standard techniques are known in the art for HLA typing, e.g., DNA typing or serological and cellular typing (Terasaki and McClleland, (1964) Nature, 204:998). One typing method for HLA identification purposes is restriction fragment length polymorphism analysis. Restriction fragment length polymorphism analysis relies upon the strong linkage between allele-specific nucleotide sequences within the exons that encode functionally significant HLA class II epitopes. Another method, PCR-SSO, relies upon the hybridization of PCR amplified products with sequence-specific oligonucleotide probes to distinguish between HLA alleles (Tiercy et al., (1990) Blood Review 4: 9-15, Saiki et al. (1989) Proc. Natl. Acad. Sci., U.S.A. 86: 6230-6234; Erlich et al. (1991) Eur. J. Immunogenet. 18(1-2): 33-55; Kawasaki et al. (1993) Methods Enzymol. 218:369-381). Yet another molecular typing method that can be used in the present invention, PCR-SSP, uses sequence specific primer amplification (Olerup and Zetterquist (1992) Tissue Antigens 39: 225-235). One of skill will also know how to type SSCP—Single-Stranded Conformational Polymorphism method. Other typing methods include high throughput methods of HLA typing. For example, one of skill will know how to amplify HLA sequences with allelic specific HLA primers and immobilize the amplification products to a solid surface using a labeled locus-specific or an allele-specific capture oligonucleotide. The presence of the oligonucleotides can then be detected and HLA allele analysis can be performed (U.S. Pat. No. 6,670,124).

For the purposes of this invention, any method of identifying which stem cell unit may be appropriate for a certain individual for transplantation can be used to type the stem cell unit.

C. Recording Stem Cell Products

The present invention provides methods of storing identifying information in connection with the stem cell products (140). Identifying information may include type identifier information, e.g., HLA type, or phenotypic description information. It may also include maintenance information. It may include information such as the volume of the stem cell unit or the total nucleated cells per unit. Identifying information may also indicate the stem cell unit as being available for a certain individual. This indication may be in the form of a name or a secret identifier code.

Each stem cell product, e.g., particular unit from an individual, will be indexed in a manner for reliable and accurate identification and retrieval. Any conventional indexing system may work in this invention as long as it is reliable and accurate. For example, each container for each stem cell product or stem cell unit may be marked with alphanumeric codes, bar codes, or any other cognizable method or combinations thereof. At a location in the bank and outside the bank, there may be an accessible and readable listing of information enabling identification of each stem cell unit and its location in the bank and enabling identification of the source and/or type of stem cell unit. This indexing system can be managed in any way known in the art, e.g., manually or non-manually, e.g. a computer and conventional software can be used.

In one embodiment of this invention, the stem cell bank comprises a system for storing a plurality of records associated with a plurality of individuals and a plurality of stem cell units. Each record may contain type information, genotypic information or phenotypic information associated with the stem cell units or specific individuals. In a specific embodiment, the system will include a cross-match table that matches types of the stem cell units with types of individuals who with to receive a stem cell unit.

In an embodiment of the invention, after the stem cell unit is recorded into the indexing system, it will be available for matching purposes, e.g., a matching program will identify an individual with matching type information and the individual will have the option of being provided the matching stem cell unit.

D. Storing Stem Cell Products

Storage of the stem cell units may be short term or long term. In some embodiments, the stored stem cells may be cryogenically preserved but any method of storing stem cell for a long duration of time may be used, e.g., including storage of cells with amino acids, inosine, adenine, etc. Any storage method may be used in this invention providing that the stored product retain viability for the therapeutic purposes discussed in this invention.

Many types of storage devices for storing stem cells are known in the art and can be used in this invention. There is no upper limit on the number of stem cell units or products than can be stored in one particular bank. In one embodiment, hundreds of stem cell products from different individuals will be stored at one bank or storage facility. In another embodiment, up to millions of products may be stored in one storage facility. In even another embodiment, there will be enough stem cell products stored in one or more storage facilities throughout the country to treat anyone in need of stem cell transplantation.

A facility for storing stem cell products may be quite small, yet still store many samples for a large number of people. In one embodiment, stem cell units products are stored in such a way to minimize the amount of space needed.

The storage facility may include means for any method of organizing, and indexing the stored products, e.g., automated robotic retrieval and/or the manipulation of stem cell units. The facility may include micromanipulation devices for processing such stem cell units. More than one storage facility may be used to store stem cell units. These facilities may each be at a different location miles apart. In some embodiments, the storage facilities may be in different states, in different countries. The storage facilities may be underground or aboveground. In some embodiments, the storage facilities are kept in secret locations unknown to the general population. The facilities may be in any protected area, including in outer space, and/or under water (including submarines).

Fault tolerance computers and redundant systems may be used throughout the storage facilities to eliminate potential problems and to provide a fail-safe system. Known conventional technologies can be used for efficient storage and retrieval of the stem cell units, e.g., Machine Vision, Robotics, Automated Guided Vehicle System, Automated Storage and Retrieval Systems, Computer Integrated Manufacturing, Computer Aided Process Planning, Statistical Process Control. Less sophisticated storage facilities may be used as well, e.g., large areas maintained at appropriate temperatures contain numerous racks on which are indexed and stored the stem cell units of the invention.

The stored stem cell units or the present invention may be available to the general public for matching purposes. In some embodiments, the stored stem cell units are available for specific groups of individuals for matching purposes, e.g., President of the United States, high government officials, Cabinet members, military.

In the event of a nuclear attack, a specific stored stem cell products may be transported from the stem cell storage facility to a separate facility, for example, to a hospital for transplantation purposes. In some embodiments, many stem cell products may be transferred to a separate facility for transplantation purposes.

A depository, storage facility or stem cell bank may include one or more storage units for storing the stem cell samples and one or more processing stations for processing the stem cell units for storage or for transplantation. Processing may be done by an inside unit or outsource unit, for example a laboratory which specializes in stem cell processing and storage. In some embodiments, the entire process, acquiring, processing, typing, recording and storing the stem cells is managed by a data processing and control unit.

### IV. Potential Recipients of Stem Cell Products

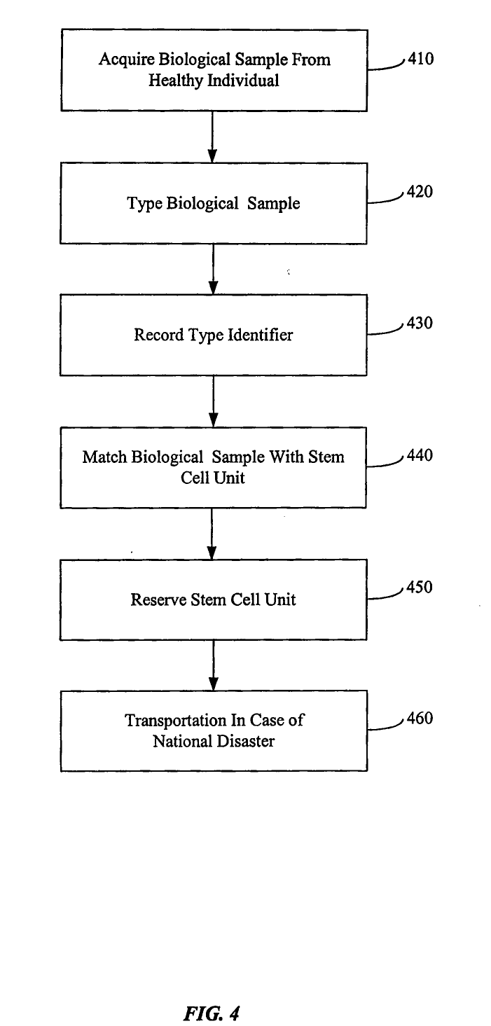

FIG. 4 is one embodiment of a process of reserving a particular stem cell unit for a potential recipient in case of lethal irradiation from a nuclear attack. It includes a method of obtaining a biological sample (410), typing the sample (420), recording the sample information (430), matching the sample with a stem cell unit (440) and reserving the stem cell unit (450) in case of lethal radiation exposure of a large numbers of persons. It optionally includes transporting the stem cell unit (460) and using the stem cell unit for transplantation.

In a particular embodiment of this invention, a biological sample, e.g., DNA sample, is obtained from an individual not yet in need of stem cell transplantation in case of future need as a result of exposure to a lethal dose of radiation, e.g., nuclear attack. In some embodiments, the individual may be any member of the general population. In other embodiments, the individual is a member of a particular group of people, e.g., government or military.

Any individual may enroll in the stem cell insurance program at any time. A typical enrollment procedure will include explaining the process to the enrollee and obtaining information from the enrollee or potential recipient. The information may include medical history information. In one specific embodiment of the invention, the enrollment process may include collecting a fee from the individual or enrolling the individual in a payment plan either at the time of enrollment in the stem cell insurance program or once a matching stem cell unit is found or both. In some embodiments of this invention, providing a matching stem cell unit for an individual will be part of an insurance plan. The insurance plan may be administered using any means known in the art.

Typically, a biological sample will be collected from the enrollee (410). The biological sample may be any sample that can be used to identify a suitable stem cell unit for that individual. For example, a biological sample can be blood or any tissue from which DNA can be prepared. In one embodiment, DNA from the potential recipient may be typed, e.g., HLA typing, according to methods known in the art in order to identify the suitable stem cell unit for that individual (420). In a specific embodiment of this invention, a type identifier is provided for each individual.

The type identifier or other information associated with the individual wanting a stem cell unit may be recorded into a system that can be used to identify an appropriate matching stem cell product, e.g., database system, indexing system, etc. (430).

Once recorded in the system, a match can be made between the type of the individual and a donor stem cell unit (440). The matching stem cell unit is available for the individual possessing the matching type identifier (450). In one embodiment of this invention, the individual's identification information is stored in connection with the stem cell unit. In some embodiments, the matched stem cell unit is then indicated as unavailable for another's use. In a particular embodiment of the invention, the match occurs before the individual is in actual need of stem cell transplantation.

In one embodiment, if the stem cell unit is needed by an individual, it may be retrieved and made available for transplantation purposes within minutes. The stem cell unit may also be further processed to prepare it for transplantation. In another embodiment, if the stem cell units are needed for a plurality of individuals, a plurality of stem cell units may be retrieved and made available for transplantation purposes within minutes or hours.

If a matching stem cell unit is not found for an individual, the individual's type identifier information, e.g., in the form of a record, will remain in the system until a match is found. When a matching stem cell unit is entered into the database, the program will indicate that a match has been found and the matching stem cell unit will be available for the individual.

In the event of a catastrophic event, e.g., nuclear attack, stored stem cell units will be immediately available for use. Stem cell transplantation may occur within days of the event, thereby providing treatment for individuals in dire need of transplantation.

In some embodiments of the invention, a method of providing a stem cell unit for insurance in case of a nuclear attack may be integrated with other health insurance schemes. For example, treatment insurance may be part of a complete insurance plan, for example, as an option in regular health insurance or as a single policy.

In some embodiments of this invention, the operating of this insurance system may be managed with the assistance of dedicated software. The software may manage enrollment of the potential recipient, acquisition of the stem cell units from donors, database management, storage monitoring, quality checking.

In some embodiments, this invention may operate on a typical computer system. The computer system may include input devices, e.g., a keyboard or mouse, a processor, e.g., a general purpose processor or a more developed processor with increased database processing capabilities, an internal memory, e.g., RAM, ROM, and external storage, e.g., disks, CD, ROMs, ASICs, External RAM, external ROM. The computer system is capable of running on any operating system.

In a particular embodiment, the database system stores information for each stem cell unit in the bank. Certain information is stored in association with each unit. The information may be associated with a particular donor, for example, an identification of the donor and the donor's medical history. Alternatively, a stem cell unit may be anonymous and not associated with a specific donor. Alternatively, or additionally, the information may be sample type information. For example, the information might include the volume of the stem cell unit or the total nucleated cells count in the product. The stored information may also include match and typing information. For example, each stem cell unit may be HLA typed and the HLA type information may be stored in association with each unit. The information stored may also be availability information. The information stored with each sample is searchable and identifies the sample in such a way that it can be located and supplied to the client immediately.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for providing a hematopoietic stem cell unit in a racially diverse allogeneic stem cell bank for a potential recipient, said method comprising:
(i) providing a plurality of stem cell units having been typed to form an allogeneic stem cell bank having a plurality of typed stem cell units;
(ii) providing a record for each of said plurality of typed stem cell units;
(iii) providing typing for a potential recipient, said potential recipient having a type identifier;
(iv) storing said record for each of said plurality of typed stem cell units and said type identifier in a database;
(v) comparing said type identifier with said record for each of said plurality of typed stem cell units to find a matched stem cell unit; and
(vi) storing said matched stem cell unit in said allogeneic stem cell bank for said potential recipient's use, thereby providing said stem cell unit for said potential recipient.

2. The method of claim 1, wherein said potential recipient is a plurality of recipients.

3. The method of claim 2, wherein said plurality of typed stem cell units has not been exposed to a lethal dose of radiation.

4. The method of claim 1, wherein said hematopoietic stem cell unit is obtained from bone marrow.

5. The method of claim 1, wherein said hematopoietic stem cell unit is obtained from peripheral blood.

6. The method of claim 1, wherein said hematopoietic stem cell unit is obtained from umbilical cord blood.

7. The method of claim 1, wherein said hematopoietic stem cell unit is obtained from placental blood.

8. The method of claim 1, wherein said each of said plurality of stem cell units are cryogenically preserved.

9. The method of claim 1, wherein the type identifier of said potential recipient is 90%, 80%, 70%, 60%, or 40% similar to said matched stem cell unit.
